(19) United States
(12) Patent Application Publication
(10) Pub. No.: US 2002/0009495 A1
(43) Pub. Date: Jan. 24, 2002

(54) Microcapsules obtainable using protein hydrolysate emulsifier

(76) Inventors: Harro Traubel, Leverkusen (DE); Hans-Albert Ehlert, Singapore (SG); Ulrich Nehen, Leverkusen (DE); Jurgen Weisser, Dormagen (DE)

Correspondence Address: BAYER CORPORATION, PATENT DEPARTMENT, 100 BAYER ROAD, PITTSBURGH, PA, 15205, US

(21) Appl. No.: 09/845,872

(22) Filed: Apr. 30, 2001

(30) Foreign Application Priority Data
May 3, 2000 (DE) 10021411.8
May 22, 2000 (DE) 10025302.4

(51) Int. Cl. A61K 7/46; A61K 9/16; A61K 9/50

(52) U.S. Cl. 424/490; 503/200; 512/4; 264/4.1

(57) ABSTRACT

The invention relates to microcapsules having walls obtained by polyaddition of polyisocyanates and polyamines in an aqueous emulsion comprising protein hydrolysate emulsifier.

### BACKGROUND OF THE INVENTION

The invention relates to microcapsules, to a process for preparing them and to their use, especially in carbonless copy papers.

Capsules for carbonless copy papers are prepared using encapsulated leucodyes capable of forming dyes on acidic surfaces (see EP 780,154). The wall material for these capsules may be polyurethaneureas, which are formed in an interfacial polyaddition process. The process is generally carried out as follows: a leucodye and at least one bifunctional isocyanate are dissolved in a hydrophobic liquid and this hydrophobic mixture is emulsified in water. The water frequently contains an emulsifier or a protective colloid, for example, partially hydrolyzed polyvinyl acetate or polyvinyl alcohols. An isocyanate-reactive amine is then added to the emulsion. A polyaddition takes place at the phase boundary to the emulsified hydrophobic droplets to form a polyurea wall around the hydrophobic droplets. Processes of this kind are described in EP 780,154, for example. These capsules are applied to the surfaces of papers using customary coating formulations.

After use, virtually all capsules pass back into the paper stock cycle via the waste paper. A waste paper recovery process separates the cellulose fibers from the coating materials and the capsule material. Gelatin and polyurethane capsules may be used as a sludge in agriculture. Paper mill sludges, provided they do not contain troublesome components, have a soil-improving effect in that polyurethaneureas and gelatin condensation products in the sludge release bioavailable nitrogen after prolonged storage: they have a fertilizing effect. Troublesome components with regard to agricultural use include, for example, polymers of vinyl-containing monomers, since these compounds degrade only very slowly, if at all. This category of compounds includes derivatives of partially hydrolyzed polyvinyl acetate or polyvinyl alcohol, which are generally used as viscosity regulators and emulsifiers in microcapsule dispersions. Similarly, the polyisocyanate capsule system amine needed for wall formation, in that it is required for polyurea formation, can have a troublesome effect on further processing, if it has not been quantitatively incorporated. More particularly, these products can have a adverse effect in the papermaker's machine when paper or paper residues where such capsules are present in the coating are recycled.

It is an object of the present invention to provide microcapsules that do not have the above-described disadvantages pertaining to recycling and yet possess the customary properties and advantages of microcapsules.

### SUMMARY OF THE INVENTION

This object is surprisingly achieved by microcapsules having walls obtained by polyaddition of at least one polyisocyanate and at least one polyamine in an aqueous emulsion comprising protein hydrolysate emulsifier.

### DETAILED DESCRIPTION OF THE INVENTION

Preferred protein hydrolysates are hydrolysates of natural proteins such as collagen hydrolysate, gelatin, or synthetic proteins. Proteins may be hydrolyzed not only by enzymatic lysis but also by alkaline hydrolysis. Alkaline hydrolysis preferentially provides products whose isoelectric pH is <6. Proteins may similarly be hydrolyzed under acidic conditions, which preferentially provides hydrolysates having an isoelectric pH range of <7.

Preference is given to protein hydrolysates that were neutralized after hydrolysis.

Preferred protein hydrolysates are obtained from untanned raw hides, tanned hides, and especially from leather shavings. The aqueous solutions of preferred protein hydrolysates have an organic content in the range from 10 to 40%, preferably from 20 to 30%, by weight. It is likewise preferable for the protein hydrolysate to have an electrical conductivity of <5,000 μS/cm.

The protein hydrolysate used is preferably used in an amount of 1 to 200%, preferably 1 to 100%, based on capsule material.

The protein hydrolysate used is preferably present as an approximately 30% aqueous solution. Particularly preferred protein hydrolysate is obtained from leather shavings, since it is generally particularly uniform. A particularly suitable protein hydrolysate is obtained from furniture leather shavings and has a solids content of about 20 to 35% by weight (preferably 25 to 32% by weight), an organic content of 20 to 30% by weight, a pH (neat) of 10 to 12, an ash content (based on solids) of 13 to 27% by weight (preferably 13 to 17% by weight), a viscosity (Brookfield, 100 rpm) of 25 to 35 mPas (preferably 27 to 32 mPas), a conductivity of <4,800 μS/cm (preferably 1,000 to 2,500 μS/cm) (1 g/l ash), and a surface tension of 50 to 60 mN/cm (preferably 55 to 60 mN/cm).

The polyisocyanates used for the capsules of the invention are preferably at least bifunctional isocyanates that on average contain at least one ester and/or amide group per mole in the main chain. These preferred isocyanates will hereinafter also be referred to as “isocyanates A”.

Microcapsules according to the invention may be prepared using, for example, isocyanates or isocyanate mixtures comprising 100 to 1% by weight of isocyanates A and 0 to 99% by weight of at least bifunctional isocyanates known for the production of microcapsules, for example, hydrophilicized polyisocyanates. By varying the ratio of the isocyanates A to customary isocyanates it is possible to adjust the properties of the microcapsules according to the invention in any desired manner, especially their mechanical strength and their hydrolysis resistance, as well as the paper engineering properties.

Preference is given to isocyanates A in which at least two isocyanate groups are attached via an organic radical that contains in the main chain at least one ester or amide group, a carbonate group, or an allophanate group or various combinations of these groups.

Preference is further given to isocyanates A, and isocyanate A-containing mixtures that contain emulsifiers. The emulsifiers may be added as such to the isocyanates (i.e., external emulsifiers). However, the emulsifiers may have been incorporated into the isocyanates. Such “emulsifier incorporation” may be obtained, for example, by reacting some of the isocyanate groups present with salt-forming and/or hydrophilicizing compounds. For example, 5 to 50% (preferably 8 to 30%) of the isocyanate groups present may be reacted in this way.

Useful salt-forming and/or hydrophilicizing compounds include, for example, dimethylolpropionic acid, N,N-dimethylethanolamine, and hydrophilic, preferably monofunctional polyethers. If desired, details pertaining to the reaction of isocyanates with salt-forming compounds may be taken from EP 564,912 or DE-A 4,418,836.

Isocyanates A are obtainable by reacting at least bifunctional isocyanates with compounds containing OH and ester, amide groups, carbonate, or allophanate groups. Such reactions are known. Useful starting isocyanates include, for example, diisocyanates such as 1,4-diisocyanatobutane, 1,6-diisocyanatohexane, 1,5-diisocyanato-2,2-dimethylpentane, 2,2,4- and 2,4,4-trimethyl-1,6-diisocyanatohexane, 1,10-diisocyanatodecane, 1,3- and 1,4-diisocyanatocyclohexane, 1-isocyanato-3,3,5-trimethyl-5-isocyanatomethylcyclohexane (“isophorone diisocyanate”), 4,4′-diisocyanatodicyclohexylmethane, 2,4- and 2,6-diisocyanato-methylcyclohexane, and mixtures thereof. In principle, aromatic isocyanates, for example, toluene diisocyanates or 4,4′-diisocyanatodiphenylmethane, may also be used. However, aliphatic isocyanates are preferred because of higher lightfastness and lower reactivity with regard to water. Polyisocyanates that are prepared by modification of the above-mentioned diisocyanates or mixtures thereof according to known processes and contain for example uretidione, urethane, isocyanurate, biuret, and/or allophanate groups may also be used as a fraction of the starting isocyanates.

Examples of useful compounds containing OH and ester and/or amide groups are products that on average contain at least two OH groups and on average at least one ester and/or amide group. Useful examples are short-chain hydroxyl-functional polyesters obtainable by esterification of diols and/or triols with dicarboxylic acids and/or dicarboxylic anhydrides or by transesterification of diols and/or triols with dicarboxylic esters of short-chain monofunctional alcohols and distillative removal of the resultant short-chain alcohols. Preferred polyesters have an average molar mass of 148 to 2,000 g/mol, preferably 148 to 1,000, especially 148 to 500 g/mol.

Useful isocyanates further include, for example, the polyisocyanates with ester groups that are obtainable by reaction of polysilyl ethers and isocyanatoalkylcarbonyl chlorides (with elimination of trimethylchlorosilane).

Useful acid components include the following compounds: dimethyl carbonate, diethyl carbonate, diphenyl carbonate, ethylene glycol carbonate, propylene glycol carbonate, oxalic and malonic diesters, succinic, glutaric and maleic acid and anhydrides thereof, adipic, sebacic, phthalic (including hydrogenated phthalic), hydroxymono- and -dicarboxylic acids (if appropriate in the form of their inner esters (i.e., lactones)) such as glycolic acid, tartaric acid, lactic acid, citric acid, hydroxycaproic acid, hydroxybutyric acid, and ricinoleic acid.

Useful diols include, for example, the following, industrially readily available diols: ethanediol, 1,2- and 1,3-propanediols, isomeric butane-, pentane-, and hexanediols, and oligo- and polymers of ethylene glycol and propylene glycol that contain ether groups. Cycloaliphatic and aromatic diols may also be mentioned but are not preferred because of the high viscosity of the esters. Useful triols include for example glycerol and trimethylolpropane and also their ethoxylation and propoxylation products.

Polyesters are obtainable, for example, by condensation of the acids and/or their esters with monofunctional alcohols and/or of the anhydrides of the acids with the recited di- and/or triols according to known processes. A narrow molecular weight distribution and hence a low viscosity and a low level of components that do not bear ester groups can be obtained by using the OH compounds in excess and subsequent extraction with water or by molecular distillation. Another viable alternative is the ring-opening transesterification of lactones (for example, butyro-, valero-, or caprolactone). This transesterification may if desired be coupled with the above-mentioned measures.

Particularly useful OH-containing compounds are obtainable by reaction of a di- or hydroxycarboxylic acid with alkylene oxide. This is a simple way of providing defined, low molecular weight ester-diols.

Suitable OH compounds that contain amide groups are preparable, for example, from the acids mentioned or esters thereof (including lactones) by reaction with hydroxyalkylamines that contain a secondary amino group. Examples of useful hydroxyalkylamines are adducts of ethylene oxide or propylene oxide with mono-C₁-C₄-alkylamines.

These last-mentioned adducts are particularly suitable, because, due to the selectivity of the amino groups, they are preparable as predominantly defined compounds. The average molecular weights of OH compounds useful for preparing isocyanates A may, for example, be in the range from 148 to 2,000. The average molecular weights are preferably 148 to 1,000, especially 148 to 500.

Isocyanates A may be prepared by reacting NCO-containing compounds with the OH-containing components in an NCO/OH ratio of 1:3 to 20:1, preferably 1.5 to 10:1, for example.

NCO/OH ratios of above 1.5:1 leave behind a considerable fraction of unconverted isocyanate, depending on the nature of the isocyanate. For industrial hygiene reasons, these free isocyanates should be removed, for example, by thin film distillation. High NCO/OH ratios are preferred because viscosity-increasing chain-extending reactions can then be substantially suppressed.

It is also possible to use esters of the hypothetical allophanic acid (known as allophanates), which can be formed by reaction of a urethane group with an isocyanate group. When the reaction of the isocyanates with the hydroxyl-containing compounds is carried out at 150° C. or higher temperatures or in the presence of catalysts (for example, hydrogen chloride gas or organic tin compounds), the urethane groups are more or less completely converted into allophanate groups, depending on the reaction time. This measure offers the advantage of obtaining products of high isocyanate content, high functionality, and low viscosity, which is of advantage for the envisaged use.

Dispersibility in water may be improved by providing the isocyanates with ionic groups (see, for example, DE-A 4,226,110) and/or with hydrophilicizing polyether chains (see, for example, DE-A 4,211,480). Useful polyethers for this purpose include, for example, monofunctional polyethers having ethylene oxide chains and an average molar mass of 220 to 2,000 g/mol (preferably 350, 550, and 850 g/mol) with methyl or ethyl end groups. Polyether addition and allophanatization may also be carried out in a single step.

As a way of facilitating emulsification in the capsule making process, the reaction of the polyisocyanates with hydrophilicizing components is preferable to mixing with external emulsifiers.

The other components required for the capsule making process, i.e., the material to be encapsulated, the hydrophobic solvent, the aqueous phase and the polyamine, conform to the prior art.

Possible materials for encapsulation include all known, preferably hydrophobic materials, for example, perfume oils, crop protection agents, reactive adhesives, and pharmaceuticals. However, preference is given to leucodyes for carbonless copy papers. Microcapsules according to the invention can also be used to obtain controlled release crop protection agents. When microcapsules according to the invention are used in the plant protection field, hydrophobic solvents used are preferably natural oils, for example, castor oil or palm oil.

When microcapsules according to the invention are used in the field of carbonless copy papers, useful leucodyes (i.e., color formers) include, for example, triphenylmethane compounds, diphenylmethane compounds, xanthene compounds, benzoxazine compounds, thiazine compounds, and spiropyran compounds, including mixed leucodyes. Useful hydrophobic solvents for this purpose include substituted biphenyls, such as secbutylbiphenyl, phenylxylylethanes, and chlorinated biphenyl, chlorinated paraffin, cotton seed oil, peanut oil, soybean oil, rapeseed oil, palm oil, tricresyl phosphate, silicone oil, dialkyl phthalates, dialkyl adipates, partially hydrogenated terphenyls, alkylated biphenyl, alkylated naphthalene, such as diisopropyinaphthalene, diaryl ethers, aryl alkyl ethers, and comparatively highly alkylated benzene, and also any desired mixtures of these hydrophilic solvents and mixtures of one or more of these hydrophobic solvents with kerosene, paraffins, and/or isoparaffins, optionally combined with extenders, by which are meant, for example, paraffin mixtures (e.g., Exxol products), isohexadecane, hydrogenated naphthenic petroleum fractions (e.g., Nytex, Gravex products), and dodecylbenzenes.

The microcapsule walls of the invention are preferably made of reaction products of the polyisocyanates mentioned above and crosslinking polyamines.

Useful polyamines include aliphatic primary and secondary polyamines. Preference is given to (poly)alkylamines, such as ethylenediamine, diethylenetriamine and its homologs, propylenediamine, piperazine, hexamethylenediamine, guanidine, optionally alkylated hydrazine derivatives, and salts. Moreover, guanidine itself or its carbonate is particularly suitable. Even water may in principle act as a crosslinker.

The quantity ratios of the individual components for microcapsule production may likewise conform to the prior art. The wall fraction is customarily 1 to 25% by weight (% of wall fraction=(mass of isocyanate+mass of oil phase)×100). For example, the particular polyamine may be used in such a ratio to the isocyanate that the equivalents of hydroxyl or amino groups amount to 50 to 100% of the equivalents of the NCO groups. The hydrophobic phase may include, for example, 0.1 to 10% by weight (preferably 1 to 8% by weight) of material to be encapsulated, 1 to 25% by weight (preferably 4 to 18% by weight) of polyisocyanates, and sufficient hydrophobic solvent to make up to 100% by weight. The weight ratio of hydrophobic phase to water phase may be for example 10:90 to 60:40, preferably 30:70 to 50:50.

The aqueous phase may include stabilizers, i.e., agents that act as protective colloids and/or viscosity-increasing agents. Examples of such agents are protein hydrolysates such as gelatin, optionally combined with polyvinyl alcohols, partially hydrolyzed polyvinyl acetate, and carboxymethylcellulose. Such agents may be present, for example, in amounts of 0.05 to 5% by weight (calculated as solids), based on the aqueous phase. Generally it is advantageous to bring microcapsule formation to completion at moderately elevated temperature, but because this interferes with biodegradation, it is preferable to do without such elevated temperatures.

Microcapsules according to the invention may be produced in customary dispersing or emulsifying apparatuses to obtain a microcapsule slurry in which the dissolved active compound is present inside small hollow microbeads. For carbonless copy papers, a slurry with or without addition of binder and/or of other auxiliaries is applied to a base paper to produce a coated back paper (“CB”). Very particular preference is given to using a binder comprising starch and/or similarly biodegradable polyurethane as are described for example in EP 824,557, EP 828,788, and EP 841,432. The CB is placed on top of a coated front paper (“CF”), which has been coated with a layer that includes a developer for the dye. Under the action of pressure, for example, due to a pencil, ball-point pen, or a typewriter character, the capsules on the CB open in those areas where pressure was exerted and the emerging leucodye comes into contact with the developer in the CF. The emerging leucodye develops into the dye in the process and reveals the pressured area as a dot, stroke, character, or the like.

The microcapsules according to the invention have a number of surprising advantages. They are more readily degradable than prior art microcapsules, for example, under conditions prevailing in de-inking processes or in external medical applications and agricultural crops. When they have been produced partly or wholly from isocyanates containing incorporated hydrophilicizing radicals, it is also possible to produce very small capsules, for example, capsules having average diameters of 1 to 10 μm.

The invention further provides a process for preparing microcapsules comprising

(a) emulsifying an oil phase comprising an organic water-immiscible isocyanate-inert solvent, the material to be encapsulated, and at least one polyisocyanate in a water phase comprising protein hydrolysates as emulsifier and optional additives, and

(b) adding to the emulsion an NH₂-containing crosslinker (polyamine) capable of reaction with isocyanate groups.

The invention further provides for the use of the microcapsules according to the invention, preferably those which encapsulate leucodyes, for preparing carbonless copy papers.

In a preferred embodiment, capsules are prepared using mixtures of 80 to 900 parts by weight of an aliphatic polyisocyanate that contains biuret groups and is based on hexamethylene diisocyanate (DESMODUR® N 3200) and 0 to 20 parts of an adduct of 70 to 87% by weight of a polyisocyanate that contains isocyanurate groups and is based on hexamethylene diisocyanate (DESMODUR® N 3300) and 13 to 30% by weight of a methanol-initiated polyethylene oxide (monofunctional, molar mass: 350 g/mol). Preferred polyamine crosslinkers in this preferred embodiment are diethylenetriamine, guanidine carbonate, and ethylenediamine, as well as NH₃. This embodiment preferably utilizes protein hydrolysates as emulsifiers and the solvent (oil phase) diisopropylnaphthalene alone or mixed with extenders.

In a particularly preferred process, color formers are dissolved in diisopropyinaphthalene with or without extenders, the isocyanate A and/or a polyisocyanate having biuret groups is or are added, and this oil phase is emulsified at 10 to 30° C. in a water phase that includes protein hydrolysate emulsifier. The emulsion is then intensively sheared to a predetermined droplet size and guanidine carbonate (10% strength aqueous solution) is added in an amount corresponding to the NCO content of the emulsion (guanidine carbonate-to-NCO ratio of 0.5:1 to 1:1) and the mixture is heated to 70 to 80° C. with stirring. After 2 to 4 hours at 70 to 80° C., the microcapsule slurry obtained is cooled and, if desired, brought with thickeners into a storage-stable form or immediately applied to a base paper.

The capsule slurry preferably has a solids content of 30 to 50% by weight. (Solids content is the content of dry capsules. Dry capsules are the fractions of the capsule dispersion that are not volatile at a drying temperature of 150° C. and atmospheric pressure).

The following examples further illustrate details for the preparation and use of the compositions of this invention. The invention, which is set forth in the foregoing disclosure, is not to be limited either in spirit or scope by these examples. Those skilled in the art will readily understand that known variations of the conditions and processes of the following preparative procedures can be used to prepare these compositions. Unless otherwise noted, all temperatures are degrees Celsius and all percentages are percentages by weight.

### EXAMPLES

### Example (Prior Art)

40 g of an isocyanate based on bis(isocyanatohexyl)oxadiazinetrione were dissolved in 360 g of a color former solution containing 345.6 g of diisopropylnaphthalene and 14.4 g of a customary color former mixture (containing 65% of PERGASCRIPT® Black PSD 134, 7% of PERGASCRIPT® Red 16B, 15% of PERGASCRIPT® Green 12 GN, and 13% of PERGASCRIPT® Blue SRB (products all obtained from Ciba-Geigy)). This solution was emulsified at 30° C. in 506.4 g of an aqueous 1% by weight polyvinyl alcohol solution (Airvol® 523, Air Products) in such a way that an emulsion was formed. This required stirring at 650 rpm. To 700 g of the emulsion thus prepared were added at room temperature 45.8 g of a 9% diethylenetriamine solution in water. The concentration of the amine solution was such that the amine equivalents introduced were exactly equivalent to the NCO equivalents of the isocyanate.

The temperature was raised to 45° C. in the course of 1 hour and then to 55° C. for 4 hours with stirring using a laboratory stirrer. This was followed by cooling back down to room temperature with stirring overnight. Thereafter, wall formation by polyaddition to polyurea was complete—NCO was no longer detectable.

The 40.8% microcapsule dispersion obtained contained capsules having an average diameter of 12 μm. This capsule dispersion was coatable onto paper in a conventional manner.

A mixture of 12.4 g of microcapsule dispersion and 20.4 g of a latex mixture (containing 1,601 g of water and 201 g of Arbocell® DE 600/30 (from J. Rettenmaier & Söhne GmbH + Co. of Ellwangen) was then used together with a KA 8588 paper binder (Bayer AG) and 22 g of water to prepare a coating composition and coated for test purposes onto an uncoated paper to prepare a CB paper (CB=coated backside) and also onto a paper coated frontside (CF) with an acidic carrier layer, to obtain a self-contained (SC) paper.

The CB paper, when subjected to the standard duplicating test, provided a copy intensity of 35% (based on nonduplicated paper).

To test the destructurability of the capsules, the SC paper was exposed to hydrolysis conditions by exposing it for 12 hours at 50° C. above ammonia vapor. After removal of the test sheet from the hydrolysis apparatus and a brief period at room temperature, the SC discolored and then had a reflectance value of 67% (based on unaged paper). This shows that the capsules did not remain stable under hydrolysis conditions. A prior art capsule prepared using bisisocyanatohexyloxadiazinetrione (the difference being that the mixer speed used in preparing the primary emulsion, which was 8,000 rpm instead of 654 rpm) showed on corresponding aging that—based on unaged SC—only 40% of the light was reflected, which is evidence of a tighter capsule after the hydrolysis test and suggests normal, inadequate degradation under de-inking conditions.

### Example 1

### Experimental Procedure

500 ml of emulsifier solution comprising 485 ml of water and 15 g of protein hydrolysate (30% strength) are initially charged with cooling. 500 ml of a solution of 20 g of color former mixture (comprising 65% of PERGASCRIPT® Black PSD 134, 7% of PERGASCRIPT® Red 16B, 15% of PERGASCRIPT® Green 12GN, and 13% of PERGASCRIPT® Blue SBR) and 35 g of a polyisocyanate containing biuret groups (DESMODUR® N 3200) in 445 ml of diisopropyinaphthalene (KMC 113) are emulsified in over 40 seconds (machine: Kotthof mixing sirene model MS16AA11G at 950 rpm, rotor/stator mixer).

This is followed by 4 minutes of further emulsification at 5,200 rpm at 20 to 25° C. 88 g of a 10% guanidinium carbonate solution are then added, and the dispersion is gradually heated to 70° C. with stirring (2 hours). After further 2 hours at 70° C., the dispersion is cooled to RT.

The dispersion is stabilized with 40 ml of thickener (2.5% of a carboxymethylcellulose thickener/6.75% of PREVENTOL D2® (preservative) in water).

Typical composition

| | | |
|---|---|---|
| Oil phase | Color former mixture | 4.0% |
| (500 ml) | Polyisocyanate | 7.0% |
| | KMC 113 solvent | 89.0% |
| | (diisopropylnaphthalene) | |
| Water phase | Protein hydrolysate | 1.0% |
| (500 ml) | emulsifier | |
| | Water | 99.0% |
| Crosslinker | Guanidinium carbonate | 10.0% |
| (88 g) | Water | 90.0% |
| Slurry | Nonvolatile constituents | 47 +/− 2.0% |

### Example 2

### Experimental Procedure

Example 1 is repeated except that a mixture of 33.25 g of the polyisocyanate containing biuret groups (DESMODUR® N 3200) and 1.75 g of a polyisocyanate that contains isocyanurate groups (Example 1 of EP 564,912) and has been hydrophilicized with 17% of polyethylene glycol monomethyl ether (molar mass 350 g/mol) is used in place of DESMODUR® N 3200.

This is followed, as in Example 1, by a further 4 minutes of emulsification at 5 200 rpm at 20 to 25° C. 88 g of a 10% guanidinium carbonate solution are then added, and the dispersion is gradually heated to 70° C. with stirring (2 hours). After further 2 hours at 70° C., the dispersion is cooled to RT.

The dispersion is stabilized with 40 ml of thickener (2.5% of a carboxymethylcellulose thickener/6.75% of PREVENTOL D2® (preservative) in water).

Typical composition

| | | |
|---|---|---|
| Oil phase | Color former mixture | 4.0% |
| (500 ml) | Polyisocyanate | 7.0% |
| | KMC 113 solvent | 89.0% |
| | (diisopropylnaphthalene) | |
| Water phase | Protein hydrolysate | 1.0% |
| (500 ml) | emulsifier | |
| | Water | 99.0% |
| Crosslinker | Guanidinium carbonate | 10.0% |
| (88 g) | Water | 90.0% |
| Slurry | Nonvolatile constituents | 47 +/− 2.0% |

What is claimed is:

1. Microcapsules having walls obtained by polyaddition of at least one polyisocyanate and at least one polyamine in an aqueous emulsion comprising a protein hydrolysate emulsifier.

2. Microcapsules according to claim 1 prepared using an at least bifunctional isocyanate containing on average at least one ester and/or amide group per mole in the main chain.

3. Microcapsules according to claim 1 prepared using an isocyanate or an isocyanate mixture containing 100 to 1% by weight of isocyanates having on average at least one ester and/or amide group per mole in the main chain and 0 to 99% by weight of at least one other bifunctional isocyanate.

4. Microcapsules according to claim 1 wherein the polyisocyanate has been partly reacted with a salt-forming and/or hydrophilicizing compound.

5. Microcapsules according to claim 4 wherein the hydrophilicizing compound contains polyether chains.

6. Microcapsules according to claim 1 wherein the microcapsule walls encapsulate a leucodye, a perfume oil, a crop protection agent, a reactive adhesive, or a pharmaceutical.

7. Microcapsules according to claim 1 prepared in the presence of a hydrophobic solvent selected from the group consisting of cotton seed oil, peanut oil, palm oil, and castor oil.

8. Method for preparing carbonless paper comprising encapsulating a leucodye in a microcapsule according to claim 1 and applying the encapsulated leucodye to a substrate.

9. Process for preparing microcapsules comprising
(a) emulsifying an oil phase comprising an organic water-immiscible isocyanate-inert solvent, a material to be encapsulated, and at least one polyisocyanate in a water phase comprising a protein hydrolysate as emulsifier with or without additives, and
(b) adding to the emulsion an NH₂-containing crosslinker (polyamine) capable of reaction with isocyanate groups.
